(19) United States
(12) Patent Application Publication
(10) Pub. No.: US 2009/0177196 A1
(43) Pub. Date: Jul. 9, 2009

(54) Medical Instrument For Rod Positioning

(75) Inventors: Stephen W. Zlock, Redding, CT (US); Carmen Walters, Hamden, CT (US)

Correspondence Address: MCCARTER & ENGLISH, LLP STAMFORD, FINANCIAL CENTRE , SUITE 304A, 695 EAST MAIN STREET, STAMFORD, CT, 06901-2138, US

(73) Assignee: APPLIED SPINE TECHNOLOGIES, INC., New Haven, CT (US)

(21) Appl. No.: 11/971,615

(22) Filed: Jan. 9, 2008

(51) Int. Cl. A61B 17/56

(52) U.S. Cl. 606/53

(57) ABSTRACT

Apparatus, systems and methods relating to manipulation, e.g. pivoting, of a rod-shaped element using a rod positioning assembly are disclosed. A rod-shaped element is initially gripped axially (relative to and) by a rod positioning assembly and then pivoted into a transverse orientation without pivoting the rod positioning assembly and/or releasing the rod-shaped element. Rod positioning assemblies include a handle element and a pair of jaw elements in communication therewith. The jaw elements are positioned distally relative to the handle element and the handle element is adapted to effect relative motion between the first and second jaw elements. The abutment faces of the jaw elements define a pair of axially aligned notches at or near the distal ends of the first and second jaw elements, a pair of traversely aligned central wells that define a circular opening that extends transversely across the jaw elements, and a pair of grooves that open up to a single transverse side of the jaw elements.

### BACKGROUND

1. Technical Field

The present disclosure is directed towards apparatus, systems and methods for implantation and manipulation of orthopedic devices. More particularly, exemplary embodiments of the present disclosure relate to apparatus, systems and methods for implantation and manipulation of an elongated member, e.g., a rods relative to an anatomical structure, e.g., the spine.

2. Background Art

In many applications, particularly those relating to orthopedic surgery and correctional techniques, elongated members are implanted into a patient interconnecting at both ends relative to one or more bones, one or more bone anchoring elements, fracture sites, and other similar locations. These elongated members may include but are not limited to support rods, pins, braces, struts and the like.

Corrective spinal surgery is an exemplary clinical application that involves implantation of elongated members, e.g., rods, pins, struts, braces or the like. Thus, for patients with certain back-related problems, e.g., spinal stenosis, scoliosis, severe back pain, and similar conditions, it may be desirable to implant a series of elongated members, e.g., spinal support rods that extend axially along the spine. In certain applications, elongated members are mounted at opposite ends within or with respect to bone-anchoring elements, e.g., pedicle screws. In this way, the elongated members extend from vertebra-to-vertebra and function to stabilize the spine. Examples of spinal correction apparatus incorporating such elongated members include Harrington implants and Steffee implants.

Often times, spinal support apparatus are designed so as to facilitate the mounting and locking of spinal support rods relative to respective bone anchoring element(s). For example, co-pending, commonly assigned U.S. patent application Ser. No. 11/818,720, which was filed on Jun. 15, 2007, and entitled “Multi Level Spinal Stabilization System,” discloses apparatus/systems wherein elongated members are mounted with respect to a patient's spine using connectors that define ball-and-socket joints. The apparatus/systems disclosed in the foregoing patent application are particularly beneficial because the disclosed ball-and-socket joints provide advantageous degrees of freedom in relative movement that are not equally attainable in static fixtures/joints. In certain of the ball-and-socket assemblies disclosed in the foregoing patent application, an elongated member is mounted with respect to a socket by inserting an enlarged ball-like head of the elongated member into a socket on a bone-anchoring element and then pivoting the member approximately 180 degrees to the correct axial positioning (relative to the spine), thereby effectively locking the ball-like head of the elongated member into the socket. The foregoing patent application is incorporated herein by reference.

Indeed, beyond the embodiments disclosed in the foregoing patent application, the process of mounting spinal support rods relative to bone-anchoring elements often requires a degree of pivoting of the rods into an axial position (relative to the spine) after an initial angled or orthogonal (relative to the spine) insertion. Furthermore, new advances in spinal correction surgery and future aspirations have sought to narrow the incision and therefore insertion area on a patient. Consequently, an angled or orthogonal (relative to the spine) insertion of a support rod may be desirable toward meeting this goal. Thus, there exists clear and apparent needs for apparatus, systems and methods that facilitate such implantation and manipulation of elongated members.

Current apparatus, systems and methods employed in the field generally involve forceps designs wherein the jaw faces define a cylindrical notch configured in size to engage a support rod around its circumference. These conventional designs carry with them many clear disadvantages. For example, since the support rod is fixed in orientation relative to such forceps designs, an operating surgeon is limited in his or her range/ability to pivot or otherwise manipulate the support rod into place. Furthermore, the act of pivoting/manipulating the forceps relative to a patient in order to position/align the support rod in a desired orientation creates added dimensions of difficulty for the surgeon in terms of controlling both movement and force. As an alternative, using a multiplicity of forceps for different holding angles would be an expensive and impractical solution. As is readily apparent, a need to switch instruments repeatedly during a surgical procedure would greatly increase the risk of error and would act to slow the procedure.

An ideal apparatus, system and method would, therefore, allow the user to pivot and/or otherwise reposition an elongated member, e.g., a support rod, without changing the angle of the apparatus (e.g., forceps) and/or releasing the elongated member/rod. As previously noted, the utility and need for such apparatus, system and method extends to a range of elongated members and into various clinical/surgical applications.

### SUMMARY OF THE DISCLOSURE

Exemplary embodiments of the present disclosure are directed primarily towards apparatus, systems and methods for implantation and manipulation of orthopedic devices and, more specifically, to apparatus systems and methods for implantation and manipulation of spinal support rods. However, while exemplary embodiments demonstrate specific application of the present disclosure to spinal procedures, other applications are herein expressly contemplated. The advantageous apparatus, systems and methods disclosed herein present new and useful means for manipulating and, more particularly, pivoting any rod-shaped element without releasing the element and/or pivoting of the apparatus within which the rod-shaped element is engaged. Indeed, the presently disclosed apparatus, systems and methods have advantageous utility in grasping and manipulating rod-shaped elements both within and outside of the medical and surgical fields. Examples of common rod-shaped elements to which the disclosure applies include but are not limited to rods, pins, screws, braces, struts, pistons, tubes, and sheaths.

The disclosed apparatus generally includes (i) a handle element positioned and configured for manipulation, (ii) a first jaw element in communication with the handle element, and (iii) a second jaw element in communication with the handle element. The first and second jaw elements are positioned distally relative to the handle element and the handle element is adapted to effect relative motion between the first and second jaw elements. Each of the jaw elements define abutment faces that are substantially aligned in a confronting relationship. Each of the first and second jaw elements define (i) an axially aligned notch formed in a distal face thereof, (ii) a central well formed in the abutment face and in communication with the axially aligned notch, the central well opening to both transverse sides thereof, and (iii) a groove in communication with the central well that opens to only a single transverse side thereof. Of note, the notch is in communication with a distal end of the central well, while the groove is in communication with the proximal end of the central well.

The first and second jaw elements are generally substantially symmetrical in design, such that the notches are aligned and together define a substantially elliptical (or eye-shaped) opening in the distally faces of the jaw elements. In a closed orientation, i.e., with the jaw elements brought into engagement by the handle element, a slight gap may exist between the distal faces of the first and second jaw elements on opposed sides of the substantially elliptical/eye-shaped opening. In addition, the substantially symmetrical design of the first and second jaw elements yields a substantially circular opening between opposed abutment faces of the jaw elements when brought into engagement due to interaction between the opposed central wells. The substantially circular opening, which extends transversely through the opposed jaw elements, is generally sized to accommodate, yet frictionally engage, a rod element for clinical use, e.g., a stabilizing rod for use in orthopedic/spinal applications.

Further, the opposed grooves of the first and second jaw elements align when the jaw elements are brought into engagement. When viewed transversely from the side on which the opposed grooves open, the opposed grooves together define a substantially tulip-shaped cavity in such side of the first and second jaw elements. The depth of the tulip-shaped cavity relative to the side walls of the jaw elements is generally greater than one-half the transverse dimension of the jaw elements, and is typically on the order of 65% to 80% of such transverse dimension. As noted previously, the opposed grooves are in communication with the substantially circular opening defined between the opposed central wells.

Methods disclosed herein generally relate to techniques for the manipulation, e.g. pivoting, and clinical positioning of a rod-shaped element using the disclosed apparatus. In general, the notches of the first and second jaw elements cooperate to grip a rod-shaped element in axial orientation within the substantially elliptical/eye-shaped opened defined therebetween. In such axial orientation, the free end of the rod-shaped element rests within the tulip-shaped cavity of the opposed first and second jaw elements. A force and/or torque may then be applied to the axially-gripped rod-shaped element, e.g., by pressing the rod shaped element against/relative to a mounting structure, e.g., a rod-receiving structure associated with an implanted pedicle screw. This force and/or torque advantageously causes the rod-shaped element to pivot to a transverse orientation within the substantially circular opening defined by the opposed central wells. In general, the rod-shaped element pivots relative to an axis defined by the notches and perpendicular to abutment faces of the jaw elements. After the rod-shaped element has pivoted to a traverse orientation, tension forces between the first and second jaw elements and/or an axial force applied to the rod-shaped element, cause the rod-shaped element to reposition into a region defined by the central wells of the first and second jaw elements.

Additional advantageous features, structures and functions of the disclosed apparatus, systems and methods will be apparent from the description which follows, particularly when read in conjunction with the appended figures.

### BRIEF DESCRIPTION OF THE DRAWINGS

To assist those of ordinary skill in the relevant art in making and using the subject matter hereof, reference is made to the appended drawings, wherein:

FIG. 1 is a perspective view of an exemplary rod positioning assembly according to the present disclosure.

FIG. 2 is a side view of the first and second jaw elements of an exemplary rod positioning assembly in a closed position.

FIG. 3 is a cross-section along line AA of FIG. 2.

FIG. 4 is a cross-section along line BB of FIG. 2.

FIG. 5 is a side view of the first and second jaw elements of an exemplary rod positioning assembly according to the present disclosure gripping an axially aligned rod-shaped element.

FIG. 6 is a cross-section along line CC of FIG. 5.

FIG. 7 is a cross-section along line DD of FIG. 5.

FIG. 8 is a side view of the first and second jaw elements of an exemplary rod introducer assembly according to the present disclosure gripping a pivoted rod-shaped element before repositioning.

FIG. 9 is a front view of the rod positioning assembly of FIG. 8.

FIG. 10 is a front view of the first and second jaw elements of an exemplary rod positioning assembly according to the present disclosure gripping a pivoted rod shaped element after repositioning.

FIG. 11 is a side view of the first and second jaw elements of an exemplary rod positioning assembly according to the present disclosure gripping an exemplary axially aligned spinal support rod.

### DESCRIPTION OF EXEMPLARY EMBODIMENT(S)

The disclosed apparatus, systems, and methods relate to manipulation, e.g. pivoting, of a rod-shaped element using an advantageous rod positioning assembly as disclosed herein. More particularly, exemplary embodiments of the disclosure involve pivoting a rod-shaped element, initially gripped axially (relative to and) by a rod positioning assembly, without the need to pivot the handle of the rod positioning assembly and/or release the rod-shaped element.

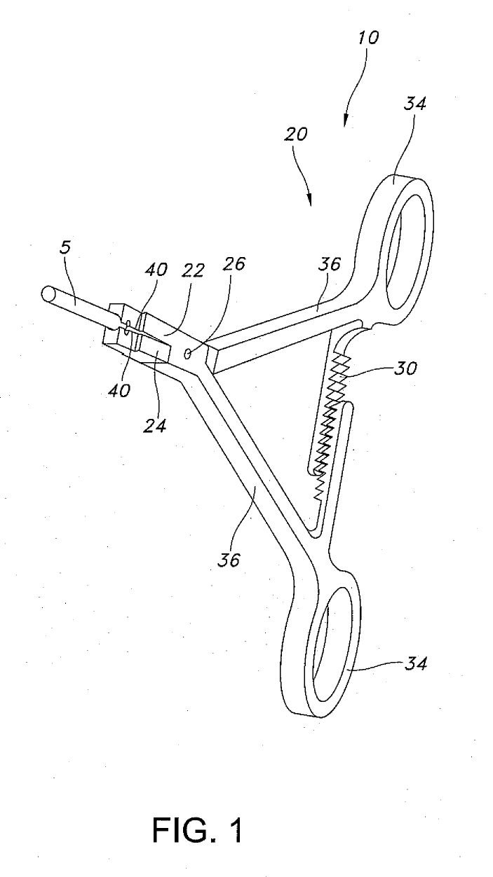

With initial reference to FIG. 1, a perspective view of an exemplary rod positioning assembly 10 is depicted gripping a rod-shaped element 5. The rod positioning assembly 10 includes a handle element 20, a first jaw element 22 and a second jaw element 24. Each of the first and second jaw elements 22 and 24 defines an abutment face 40. The first and second jaw elements 22 and 24 are positioned distally relative to the handle element 20 and are adapted to be moved relative to each other through manipulation of the handle element 20. In exemplary embodiments, the handle element 20 may include a pair of forcep arms 36 and gripping handles 34. Alternative handle elements may be employed without departing from the spirit or scope of the present disclosure, e.g., pistol-shaped handle elements as are known in the art.

The handle element 20 is adapted to effect relative motion between the first and second jaw elements 22 and 24. Generally, the handle element 20 is adapted to effect an opening or closing motion relative to the abutment faces 40 of the first and second jaw elements 22 and 24. In exemplary embodiments, the opening or closing motion is effected by pivoting the jaw elements 22 and 24 around the axis defined by a pin 26. A cooperative locking element 30, e.g. cooperating ratchet teeth, a clamp mechanism, a pin mechanism or the like, may be used to lock the first and second jaw elements in place once a desired relative position is achieved.

The jaw elements 22 and 24 pivot relative to a pivot pin 26 positioned in space relation relative to the distal ends thereof. In exemplary embodiments, the locking element 30 and handle element 20 may be used to effect and maintain tension/compression forces between the first and second jaw elements 22 and 24, e.g., when engaging a rod-shaped element 5 therebetween. Generally, the first and second jaw elements 22 and 24 exhibit a level of elasticity based at least in part on the length of the jaw elements distal to the pivot pin 5. Through relative positioning of pivot pin 26 relative to the overall size/dimension of rod positioning assembly 10 and the selection of materials of construction, a desired level of elasticity may be achieved for jaw elements 22 and 24. Rod positioning assembly 10 is generally fabricated, in whole or in part, from stainless steel. Thus, first and second jaw elements 22 and 24 are able to receive and accommodate substantially rigid members therebetween, e.g., rod-shaped element 5 that is characterized by a diameter that exceeds the corresponding openings in rod positioning assembly 10.

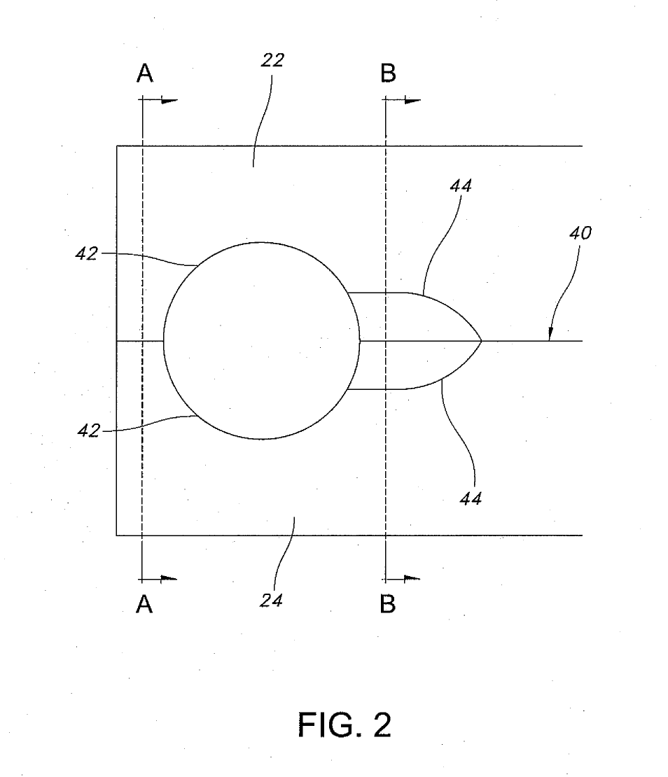

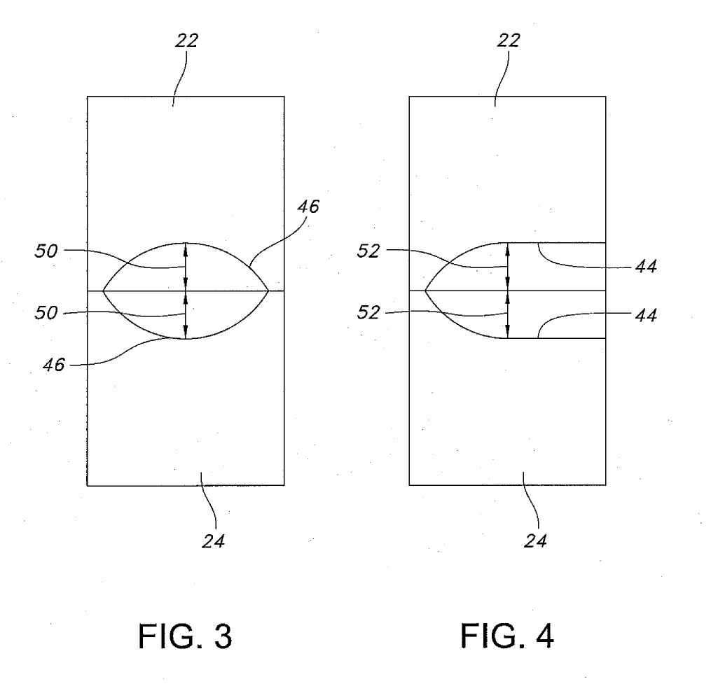

With reference to FIGS. 2-4, portions of the first and second jaw elements 22 and 24 of an exemplary rod positioning assembly 10 are schematically depicted. FIG. 2 depicts a side view of the distal ends of first and second jaw elements 22 and 24 of exemplary rod positioning assembly 10. FIG. 3 depicts a cross-section of the distal ends of first and second jaw elements 22 and 24 taken along line AA of FIG. 2. FIG. 4 depicts a cross-section of first and second jaw elements taken an intermediate location along line BB of FIG. 2. In FIGS. 2-4, the rod positioning assembly 10 is depicted in a closed position defined by the positioning of the abutment faces 40 adjacent to one another. In exemplary embodiments, the abutment face 40 of the first jaw member 22 substantially mirrors the abutment face 40 of the second jaw member 24. Alternatively, non-mirroring jaw configurations may be preferred, e.g., for use with asymmetric rod-shaped elements.

In general, each abutment face 40 includes: (i) a notch 46, (ii) a central well 42, and (iii) a groove 44. The notches 46 are typically axially aligned with the rod positioning assembly 10 and defined relative to the distal end of the first and second jaw elements 22 and 24. The central wells 42 are typically traversely aligned with the rod positioning assembly 10 and define a region that extends through both traverse sides of the first and second jaw elements 22 and 24. The grooves 44 are typically axially aligned with the rod positioning assembly 10 and define a region that opens up to a single transverse side of the first and second jaw elements 22 and 24. Generally, each notch 46 is in communication with the distal side of the corresponding central well 42 and each groove 44 is in communication with the proximal side of the corresponding central well 42.

The opposed notches 46 associated with the first and second jaw elements 22, 24 cooperate to define a substantially elliptical or eye-shaped opening therebetween. Such elliptical/eye-shaped opening advantageously defines and/or provides means for gripping a rod-shaped member, e.g., rod-shaped element 5, in an axial position/orientation relative to the rod positioning assembly 10. Thus, in exemplary embodiments of the present disclosure, the opposed notches 46 are arc-shaped with arch radii substantially similar to the radius of rod-shaped elements to be received therewithin. The depths 50 of the notches 46 are typically configured to be on the order of about half the radius of rod-shaped elements to be received therewithin.

The present disclosure, however, is not limited by or to this particular relative configuration and/or dimensional relationship. Indeed, decreasing the depths 50 and/or increasing the radii of one or both notches 46 may be accommodated; a decrease of depth 50 and/or an increase in the radii of notch(es) 46 (relative to the typical dimensional range of “on the order of about half the radius of rod-shaped elements to be received therewithin”) generally translates to a reduction in the rotational resistance delivered/applied to a held rod-shaped element and consequently a reduction in the force necessary to pivot the rod-shaped element toward a traverse orientation. Similarly, increasing the depths 50 and/or decreasing the radii of one or both notches 46 may be accommodated; an increase of depth 50 and/or an decrease in the radii of notch(es) 46 (relative to the typical dimensional range of “on the order of about half the radius of rod-shaped elements to be received therewithin”) generally translates to an increase in rotational resistance delivered/applied to a held rod-shaped element and therefore an increase in the required pivotal force. Thus, varying the configuration of the arc-shaped notch(es) 46 may be desirable for producing rod positioning assemblies 10 that deliver a desired force level to a rod-shaped element positioned therebetween and/or to provide desirable tolerance levels as between opposed jaw elements 22 and 24.

In exemplary embodiments of the present disclosure, the axial lengths of the notches 44 are configured and dimensioned to approximate between about ½ and ¼ of the diameter of the rod-shaped elements to be positioned therebetween. Of note, the axial lengths of the notches 44 generally effects the overall stability of a rod-shaped element gripped therebetween. Thus, increased axial lengths of notches 44 generally increases grip strength/stability that is delivered/applied to a rod-shaped element positioned therebetween.

The grooves 44 generally define a tulip-shaped opening in a side face of jaw elements 22 and 24. The grooves 44 function to stabilize the proximal end of an axially gripped rod-shaped element positioned between jaw elements 22 and 24. The grooves 44 also establish and guide the rotational direction of the rod-shaped element when pivoted relative to rod positioning assembly 10, wherein the proximal end of the rod-shaped element is permitted to pivot outward relative to the open transverse face of the grooves 44. In exemplary embodiments, the tulip-shaped opening defined by grooves 44 is dimensioned and configured so as approximate the size/shape of the proximal end of a gripped rod-shaped element. Thus, in exemplary embodiments of the present disclosure, the grooves 44 are formed by tapered arc-shaped walls, wherein the arc radii substantially match the radius of rod-shaped elements to be positioned therewithin. The depths 52 of the grooves 44 are generally configured so as to not impede a rod-shaped element's rotational movement. Of note, the grooves 44 are positioned proximal relative to notches 46 and, therefore, the elastic deflection of jaw elements 22 and 24 when gripping a rod-shaped element will be of a lesser amount in the region of grooves 44 as compared to notches 46. In exemplary embodiments, the axial lengths of the grooves 44 may be varied, e.g., to effect the extent of the engagement region for interaction with a rod-shaped element resting thereon/thereagainst.

The central wells 42 define a substantially circular opening that extends transversely through the jaw elements 22 and 24 when the jaw elements are brought into engagement with each other. The disclosed rod positioning assembly 10 advantageously permits a rod-shaped element to be pivoted/repositioned within the substantially circular opening defined by central wells 42. As such, the rod-shaped element assumes a traverse orientation relative to the rod positioning assembly 10, without the need to pivot/reposition the handle element 20 and/or to open the jaw elements 22 and 24 to permit repositioning of the rod-shaped element relative thereto. In exemplary embodiments, each central well 42 is substantially semicircular in shape with a radius that is slightly smaller than the diameter of rod-shaped elements to be positioned therewithin. Thus, a transversely oriented rod-shaped element is generally firmly gripped by the central wells 42 when the first and second jaw elements of the rod positioning assembly 10 are in the closed position.

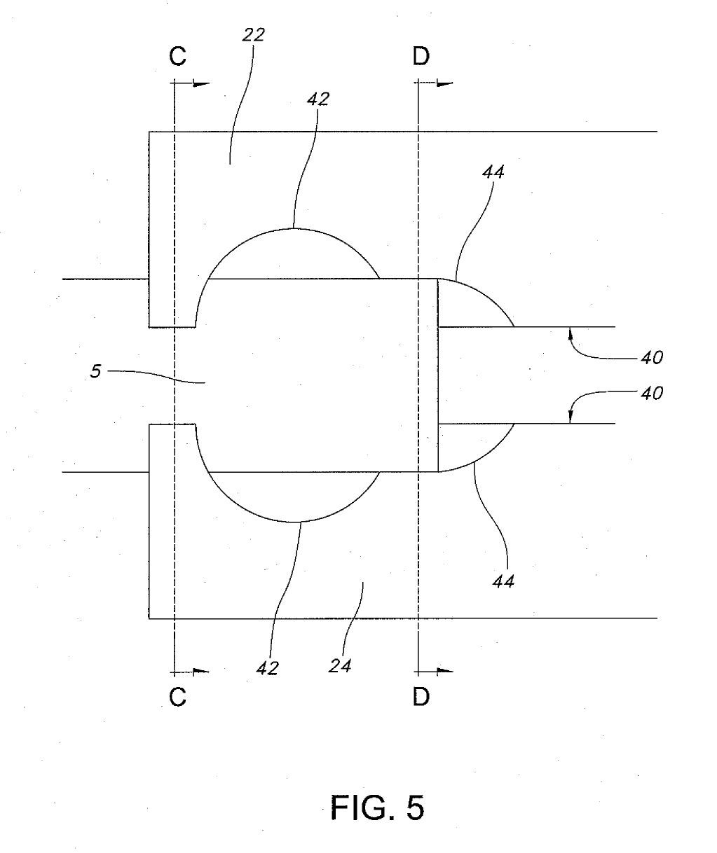

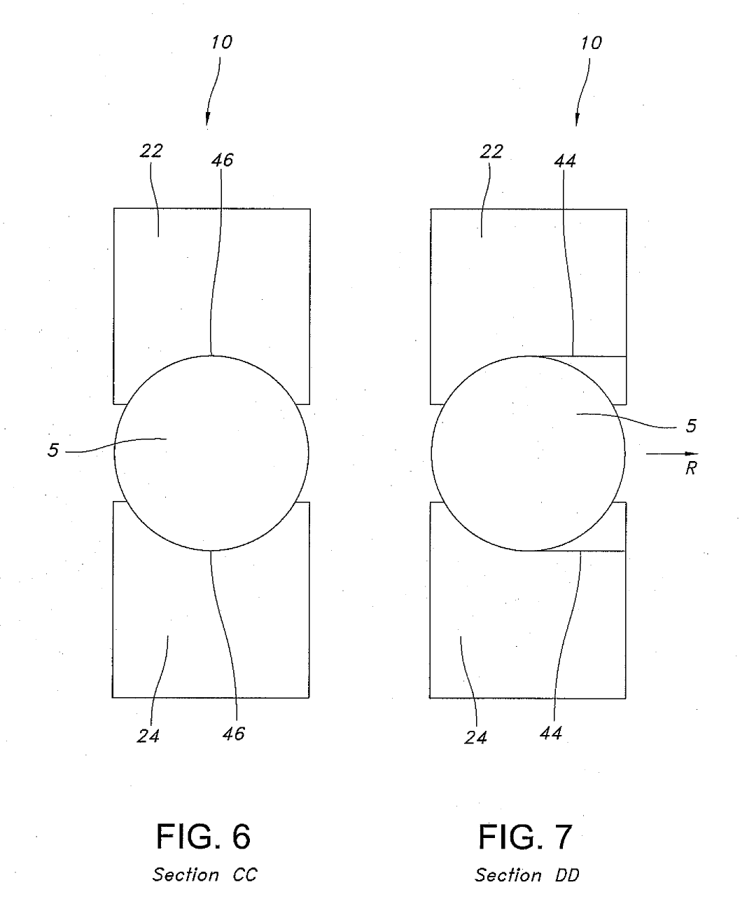

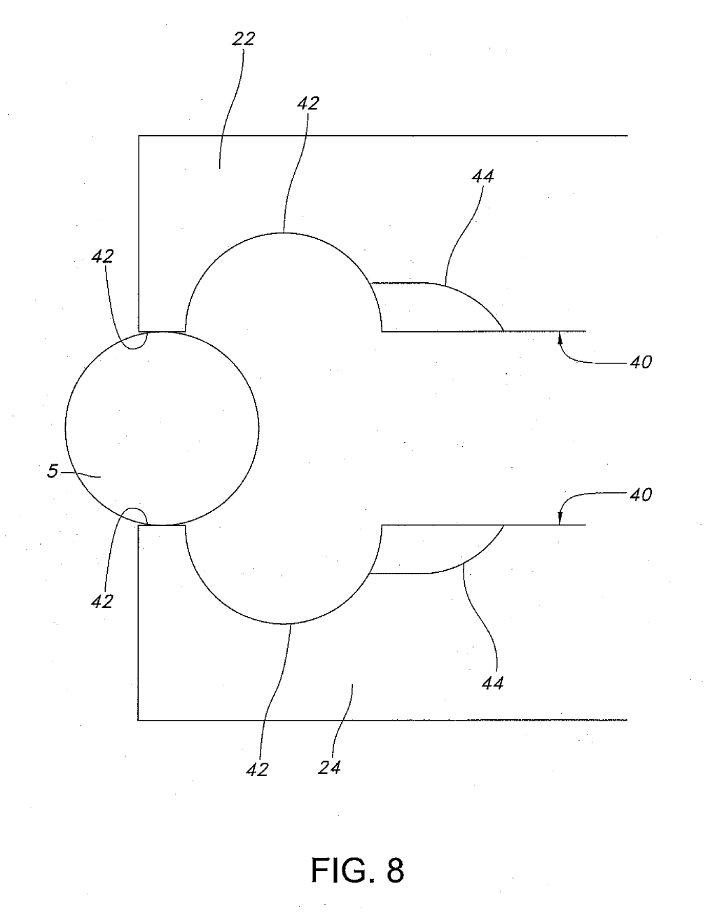

With reference to FIGS. 5-7, the first and second jaw elements 22 and 24 of an exemplary rod positioning assembly 10 are schematically depicted gripping an axially-aligned rod-shaped element 5. FIG. 5 depicts a side view of the rod positioning assembly 10. FIG. 6 depicts a cross-section of the distal end of the jaw elements 22, 24 positioned around rod-shaped element 5 taken along line CC of FIG. 5. FIG. 7 depicts a cross-section of the jaw elements 22, 24 taken along line DD of FIG. 5 in the region of grooves 44. As shown in FIGS. 5-6, the rod-shaped element 5 is gripped by the opposed notches 46 of the jaw elements 22, 24. The cooperating grooves 44 of the jaw elements 22, 24 support (but do not grip) the proximal end of the rod-shaped element 5. The open transverse face defined by the grooves 44 determines the rotational direction “R” for the rod-shaped element 5 when moving between an axial orientation and a transverse orientation relative to rod positioning assembly 10. The central wells 42 do not effect/engage the rod-shaped element 5 while it is axially oriented. Indeed, the rod-shaped element 5 spans the central wells 42 when axially oriented.

In general, the rod-shaped element 5 is pivoted from its axial orientation to its transverse orientation by applying torque to the exposed distal end thereof. In exemplary implementations, the rod-shaped element 5 may be torqued by applying a traverse force at or near the distal end of the rod-shaped element 5, e.g., by pressing such distal end against another structure, such as a mounting structure associated with a pedicle screw.

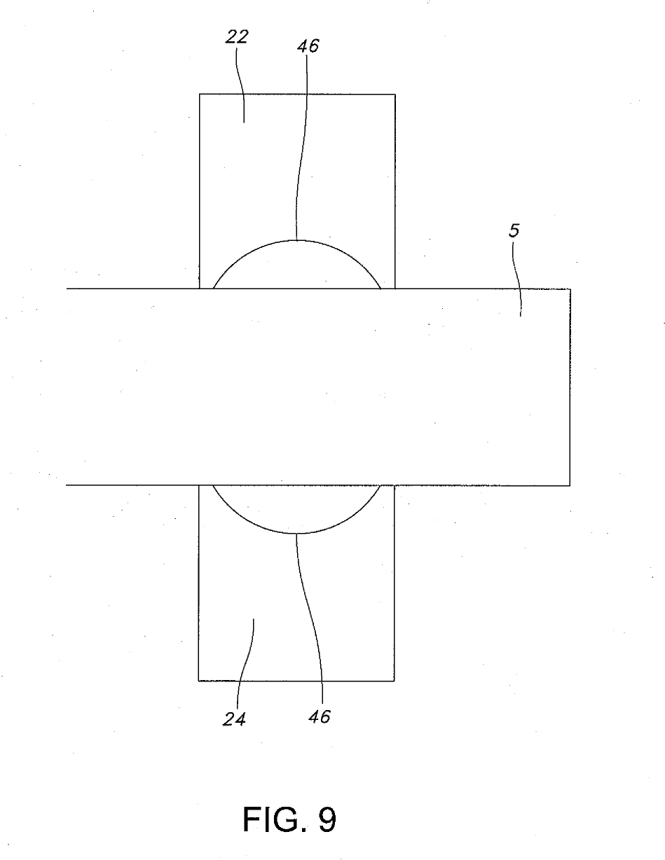

With reference to FIGS. 8-9, further side and front views of the distal portions of first and second jaw elements 22 and 24 of an exemplary rod positioning assembly 10 are schematically depicted. With initial reference to FIG. 8, the first and second jaw elements 22 and 24 initially grip a rod-shaped element 5 within the distal edges 42 of abutment faces 40, i.e., adjacent notches 46, as the rod-shaped element pivots from its axial orientation to its transverse orientation. Generally, the distal edges 42 of first and second jaw elements 22 and 24 exert compression forces on the rod-shaped element 5 in the transitional positioning schematically depicted in FIG. 8. These compressive forces and the overall geometry of the jaw elements 22, 24 advantageously cause the rod-shaped element to reposition itself into the substantially circular opening defined by the cooperating central wells 42 of the respective jaw elements.

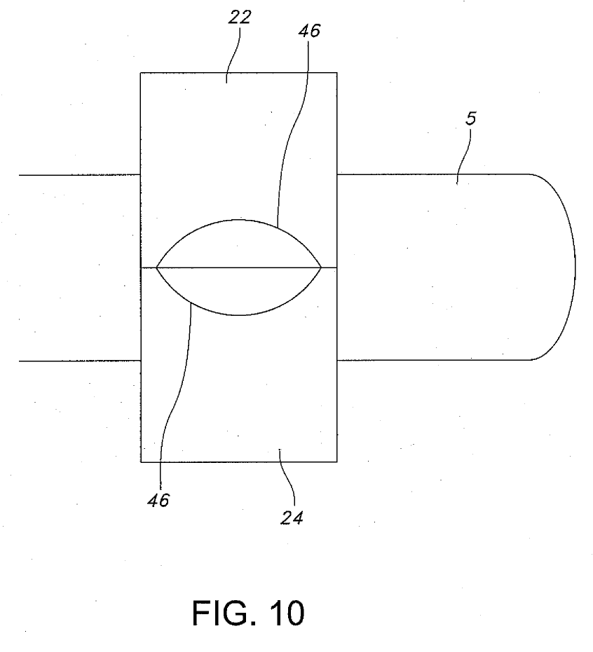

In exemplary embodiments of the present disclosure, the first and second jaw elements 22 and 24 are outwardly deflected by the interplay between the diameter of the rod-shaped element relative to the diameter of the substantially circular opening defined by the cooperating central wells 42. The relatively short axial length of the notch elements 46 combined with the increased compressive forces, increases the propensity of the rod-shaped element 5 to reposition into a more stable region. FIG. 10 depicts a front view of the first and second jaw elements 22 and 24 of an exemplary rod positioning assembly 10 gripping a rod-shaped element 5 after it has assumed a substantially transverse orientation within the substantially circular opening defined by cooperating central wells 42.

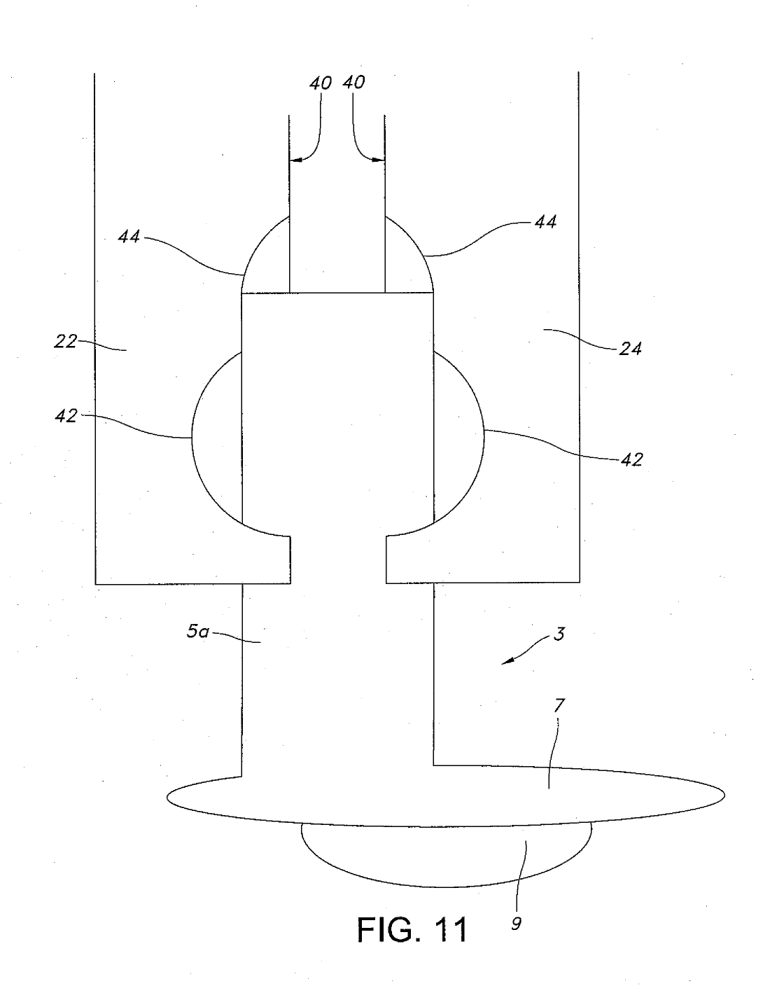

With reference now to FIG. 11, a side view of the first and second jaw elements 22 and 24 of an exemplary rod positioning assembly 10 gripping an exemplary axially aligned spinal support rod assembly 3 is depicted. The exemplary spinal support rod assembly 3 includes a rod-shaped element 5a, a plate element 7 and a base element 9. The rod-shaped element 5a is positioned off-center relative to the plate element 7 and the base element 9. Thus, the necessary torque to cause pivoting/rotation of support rod assembly 3 may be effected through interaction with support rod element 5a, base element 9, plate element 7 or a combination thereof.

The apparatus, systems and methods of the present disclosure provide highly advantageous positioning and manipulation functionalities with respect to rod-shaped elements in clinical settings. A medical practitioner, e.g., a surgeon, is able to introduce a rod-shaped element to a clinical/anatomical location with the rod-shaped element securely gripped in a substantially axial orientation relative to the instrument/apparatus that he/she is holding, and to reliably and efficiently rotate/pivot the rod-shaped element into a transverse orientation relative to the instrument/apparatus without rotation of the instrument/apparatus and/or release and re-gripping of the rod-shaped element. Of note, the rod-shaped element may have various features, functions and/or structures that are defined and/or appended to the distal end thereof without interfering with the operation and/or efficacy of the disclosed apparatus/instrument and associated methods.

Although the present disclosure is described with reference to exemplary embodiments and implementations thereof, the present disclosure is not to be limited by or to such exemplary embodiments and/or implementations. Rather, the apparatus, systems, and methods of the present disclosure are susceptible to various modifications, variations and/or enhancements without departing from the spirit or scope of the present disclosure. Accordingly, the present disclosure expressly encompasses all such modifications, variations and enhancements within its scope.

1. A rod positioning assembly, comprising
a. a handle element,
b. a first jaw element in operative communication with the handle element, and
c. a second jaw element in operative communication with the handle element,
wherein each of the first and second jaw elements define an abutment face that includes: (i) a notch defined relative to the distal end of the first and second jaw elements, (ii) a central well in communication with the notch that extends across the abutment face of the jaw element, and (iii) a groove in communication with the central well that opens up to a single transverse side of the jaw element;
wherein a rod-shaped element that is gripped between opposed notches of the first and second jaw elements in an axial orientation such that a proximal end of the rod-shaped element rests upon cooperating grooves of the first and second jaw elements is caused to pivot into a transverse orientation between cooperating central wells of the first and second jaw members through application of a torque to the rod-shaped element.

2. The rod positioning assembly of claim 1, wherein the abutment face of the first jaw element substantially mirrors the abutment face of the second jaw element.

3. The rod positioning assembly of claim 1, wherein the notches are arc-shaped.

4. The rod positioning assembly of claim 3, wherein the radii of the arc-shaped notches are substantially similar to the radius of the rod-shaped element.

5. The rod positioning assembly of claim 1, wherein the depths of the notches are approximately equal to half the radius of the rod-shaped element.

6. The rod positioning assembly of claim 1, wherein the configuration of the notches is selected so as to achieve a desired force tolerance.

7. The rod positioning assembly of claim 1, wherein the grooves are configured so as to approximate the shape and size of the proximal end of the rod-shaped element.

8. The rod positioning assembly of claim 1, wherein the central wells are semicircular in shape.

9. The rod positioning assembly of claim 8, wherein radii of the central wells are slightly smaller than the radius of the rod shaped element.

10. The rod positioning assembly of claim 1, wherein the axial lengths of the notches are approximately one-half to one-quarter of the diameter of the rod-shaped element.

11. The rod positioning assembly of claim 1, wherein the first and second jaw elements are adapted to deflect when brought into engagement with a rod-shaped element.

12. The rod positioning assembly of claim 1, wherein the first and second jaw elements are positioned distally relative to the handle element.

13. The rod positioning assembly of claim 1, wherein the handle element is adapted to effect relative motion between the first and second jaw elements.

14. A method for manipulating a rod-shaped element, the method comprising:
a. providing a rod positioning assembly that includes (i) a handle element, (ii) a first jaw element in operative communication with the handle element, and (iii) a second jaw element in operative communication with the handle element, wherein each of the first and second jaw elements define an abutment face that includes (i) a notch defined relative to the distal end of the first and second jaw elements, (ii) a central well in communication with the notch that extends across the abutment face of the jaw element, and (iii) a groove in communication with the central well that opens up to a single transverse side of the jaw element;
b. manipulating the handle element in order to grip a rod shaped element in an axial orientation between the notches of the first and second jaw elements;
c. applying a force relative to the rod-shaped element in order to pivot the rod-shaped element to a transverse orientation, wherein the rod-shaped element automatically positions itself between cooperating central wells of the first and second jaw elements.

15. The method according to claim 14, wherein the rod-shaped element is a spinal support rod.

16. The method according to claim 14, further comprising manipulating the transversely oriented rod-shaped element before releasing it from between the first and second jaw elements.

17. The method according to claim 14, further comprising locking the first and second jaw elements to grip the rod-shaped element.

18. The method according to claim 14, wherein a distal end of the rod-shaped element is positioned in a mounting structure of a pedicle screw before applying the pivoting force thereto.
